(19) United States
(12) Patent Application Publication
(10) Pub. No.: US 2015/0344872 A1
(43) Pub. Date: Dec. 3, 2015

(54) METHODS OF SYNTHESIS OF OLIGONUCLEOTIDE TAGGED COMBINATORIAL LIBRARIES AND USES THEREOF

(71) Applicant: DICE Molecules SV, LLC, Menlo Park, CA (US)

(72) Inventors: Pehr Harbury, Portola Valley, CA (US); Madan Paidhungat, San Francisco, CA (US); Phillip Patten, Portola Valley, CA (US); Richard Edward Watts, Palo Alto, CA (US)

(73) Assignee: DICE Molecules SV, LLC, Menlo Park, CA (US)

(21) Appl. No.: 14/726,478

(22) Filed: May 30, 2015

Related U.S. Application Data
(60) Provisional application No. 62/006,838, filed on Jun. 2, 2014.

(51) Int. Cl. C12N 15/10; C12Q 1/68

(52) U.S. Cl. CPC C12N 15/1065; C12Q 1/6874

(57) ABSTRACT

Provided herein are methods of synthesis of a compound which includes a functional moiety operatively linked to a tagging oligonucleotide, libraries thereof and methods of using the compound and libraries thereof to identify compounds which bind to a target.

### CROSS-REFERENCE TO RELATED APPLICATION

This application claims priority under 35 U.S.C. §119(e) from U.S. Provisional Application Ser. No. 62/006,838 filed Jun. 2, 2014, which are hereby incorporated by reference in their entirety.

### FIELD

Provided herein are methods of synthesis of a compound which includes a functional moiety operatively linked to a tagging oligonucleotide, libraries thereof and methods of using the compound and libraries thereof to identify compounds which bind to a target.

### BACKGROUND

Oligonucleotide tagged combinatorial libraries were first described in the in the early nineties as methods for drug discovery (Dower et al., U.S. Pat. No. 6,140,493; Lerner et al., U.S. Pat. No. 6,060,596; Dower et al., U.S. Pat. No. 5,789,162; Lerner et al., U.S. Pat. No. 5,723,598; Dower et al.; U.S. Pat. No. 5,708,153; Dower et al., U.S. Pat. No. 5,639,603; and Lerner et al., U.S. Pat. No. 5,573,905). However, until new methods for making oligonucleotide tagged combinatorial libraries were developed (e.g., Harbury, et al., U.S. Pat. No. 7,479,472; Liu et al., U.S. Pat. No. 7,070,928; Liu et al., U.S. Pat. No. 7,223,545; Liu et al., U.S. Pat. No. 7,442,160; Liu et al., U.S. Pat. No. 7,491,160; Liu et al., U.S. Pat. No. 7,557,068; Liu et al., U.S. Pat. No. 7,771,935; Liu et al., U.S. Pat. No. 7,807,408; Liu et al., U.S. Pat. No. 7,998,904; Liu et al., U.S. Pat. No. 8,017,323; Liu et al., U.S. Pat. No. 8,183,178; Pedersen et al., U.S. Pat. No. 7,277,713; Pedersen et al., U.S. Pat. No. 7,413,854; Gouliev et al., U.S. Pat. No. 7,704,925; Franch et al., U.S. Pat. No. 7,915,201; Gouliev et al., U.S. Pat. No. 8,722,583; Freskgard et al., U.S. Patent Application No. 2006/0269920; Freskgard et al., U.S. Patent Application No. 2012/0028812; Hansen et al., U.S. Pat. No. 7,928,211; Hansen et al., U.S. Pat. No. 8,202,823; Hansen et al., U.S. Patent Application No. 2013/0005581; Hansen et al., U.S. Patent Application No. 2013/0288929; Neri et al., U.S. Pat. No. 8,642,514; Neri et al., U.S. Pat. No. 8,673,824; Neri et al., U.S. Patent Application No. 2014/01288290; Morgan et al., U.S. Pat. No. 7,972,992; Morgan et al., U.S. Pat. No. 7,935,658; Morgan et al., U.S. Patent Application No. 2011/0136697; Morgan et al., U.S. Pat. No. 7,972,994; Morgan et al., U.S. Pat. No. 7,989,395; Morgan et al., U.S. Pat. No. 8,410,028; Morgan et al., U.S. Pat. No. 8,598,089; Morgan et al., U.S. patent application Ser. No. 14/085,271; Wagner et al., U.S. Patent Application No. 2012/0053901; and Keefe et al., U.S. Patent Application No. 2014/0315762) few compounds derived from these libraries were identified which led to useful drug candidates.

However, more recently, numerous interesting leads have been identified against targets of intense biological interest (e.g., Kollmann et al., Biorg. Med Chem. (2014) http//\\://dx.doi.org./10.1016/j.bmc.201401.050; Disch et al., J. Med. Chem. 2013, 56, 3666; Podolin et al., Prostaglandins & Other Lipid Mediators, 104-105, (2013) 25; Clark et al., U.S. Pat. No. 8,119,798) which has lead to renewed interest in oligonucleotide tagged combinatorial libraries.

Accordingly, what is needed are new methods for making and using oligonucleotide tagged combinatorial libraries.

### SUMMARY

The present invention satisfies these and other needs by providing a method for synthesis of a compound which includes a functional moiety operatively linked to a tagging oligonucleotide, libraries thereof and methods of using the compound and libraries thereof to identify compounds which bind to a target.

In one aspect, a method of synthesizing a compound including a functional moiety operatively linked to a tagging oligonucleotide is provided. The method includes the steps of splitting an initiator compound including a functional group operatively linked to a tagging oligonucleotide into multiple fractions; reacting the initiator compound with a unique oligonucleotide tag; optionally, hybridizing the initiator compound with a complementary oligonucleotide; non-covalently immobilizing the initiator compound on a substance; reacting the functional group of the initiator compound with a unique building block to provide the functional moiety; eluting the functional moiety from the substance; and combining the multiple fractions.

In another aspect, a method of synthesizing a compound including a functional moiety operatively linked to a tagging oligonucleotide is provided. The method includes the steps of splitting an initiator compound including a functional group operatively linked to a tagging oligonucleotide into multiple fractions; optionally, hybridizing the initiator compound with a complementary oligonucleotide; non-covalently immobilizing the initiator compound on a substance; reacting the functional group of the initiator compound with a unique building block to provide the functional moiety; eluting the functional moiety from the substance; and combining the multiple fractions.

In still another aspect, a method of synthesizing a compound including a functional moiety operatively linked to a tagging oligonucleotide is provided. The method includes the steps of splitting an initiator compound including a functional group operatively linked to a oligonucleotide into multiple fractions; non-covalently immobilizing the initiator compound on a substance; reacting the functional group of the initiator compound with a unique building block to provide the functional moiety; eluting the functional moiety from the substance; reacting the functional moiety with a unique tagging oligonucleotide; optionally, hybridizing the functional moiety with a complementary oligonucleotide; and combining the multiple fractions.

In still another aspect, a method of synthesizing a library of compounds wherein each compound includes a functional moiety including two or more building blocks operatively linked to a tagging oligonucleotide is provided. The method includes (a) splitting a functional moiety operatively linked with an oligonucleotide into multiple fractions; (b) reacting the functional moiety with a unique tagging oligonucleotide; (c) optionally, hybridizing the functional moiety with a complementary oligonucleotide; (d) non-covalently immobilizing the functional moiety on a substance; (e) reacting the functional moiety with a unique building block; (f) eluting the functional moiety from the ion exchange substance; (g) combining the multiple fractions; and optionally, repeating steps a-g, j times wherein j is an integer greater than or equal to 1. In the above method, the functional moiety can be reacted with the unique oligonucleotide tag before reaction of the functional moiety with a building block or the functional moiety can be reacted with a building block before reaction of the functional moiety with the unique oligonucleotide tag.

In still another aspect, a method of synthesizing a compound including a functional moiety operatively linked to a tagging oligonucleotide is provided. The method includes the steps of splitting an initiator compound including a functional group operatively linked to a tagging oligonucleotide into multiple fractions; reacting the initiator compound with a unique oligonucleotide tag; optionally, hybridizing the initiator compound with a complementary oligonucleotide; covalently immobilizing the initiator compound on a substance; reacting the functional group of the initiator compound with a unique building block to provide the functional moiety; eluting the functional moiety from the substance; and combining the multiple fractions.

In still another aspect, a method of synthesizing a compound including a functional moiety operatively linked to a tagging oligonucleotide is provided. The method includes the steps of splitting an initiator compound including a functional group operatively linked to a tagging oligonucleotide into multiple fractions; optionally, hybridizing the initiator compound with a complementary oligonucleotide; covalently immobilizing the initiator compound on a substance; reacting the functional group of the initiator compound with a unique building block to provide the functional moiety; eluting the functional moiety from the substance; and combining the multiple fractions.

In still another aspect, a method of synthesizing a compound including a functional moiety operatively linked to a tagging oligonucleotide is provided. The method includes the steps of splitting an initiator compound including a functional group operatively linked to a oligonucleotide into multiple fractions; covalently immobilizing the initiator compound on a substance; reacting the functional group of the initiator compound with a unique building block to provide the functional moiety; eluting the functional moiety from the substance; reacting the functional moiety with a unique tagging oligonucleotide; optionally, hybridizing the functional moiety with a complementary oligonucleotide; and combining the multiple fractions.

In still another aspect, a method of synthesizing a library of compounds wherein each compound includes a functional moiety including two or more building blocks operatively linked to a tagging oligonucleotide is provided. The method includes (a) splitting a functional moiety operatively linked with an oligonucleotide into multiple fractions; (b) reacting the functional moiety with a unique tagging oligonucleotide; (c) optionally, hybridizing the functional moiety with a complementary oligonucleotide; (d) covalently immobilizing the functional moiety on a substance; (e) reacting the functional moiety with a unique building block; (f) eluting the functional moiety from the ion exchange substance; (g) combining the multiple fractions; and optionally, repeating steps a-g, j times wherein j is an integer greater than or equal to 1. In the above method, the functional moiety can be reacted with the unique oligonucleotide tag before reaction of the functional moiety with a building block or the functional moiety can be reacted with a building block before reaction of the functional moiety with the unique oligonucleotide tag.

In still another aspect, a method of synthesizing a compound including a functional moiety operatively linked to a tagging oligonucleotide is provided. The method includes the steps of splitting an initiator compound including a functional group operatively linked to a tagging oligonucleotide into multiple fractions; reacting the initiator compound with a unique oligonucleotide tag; optionally, hybridizing the initiator compound with a complementary oligonucleotide; physically immobilizing the initiator compound on a substance; reacting the functional group of the initiator compound with a unique building block to provide the functional moiety; eluting the functional moiety from the substance; and combining the multiple fractions.

In still another aspect, a method of synthesizing a compound including a functional moiety operatively linked to a tagging oligonucleotide is provided. The method includes the steps of splitting an initiator compound including a functional group operatively linked to a tagging oligonucleotide into multiple fractions; optionally, hybridizing the initiator compound with a complementary oligonucleotide; physically immobilizing the initiator compound on a substance; reacting the functional group of the initiator compound with a unique building block to provide the functional moiety; eluting the functional moiety from the substance; and combining the multiple fractions.

In still another aspect, a method of synthesizing a compound including a functional moiety operatively linked to a tagging oligonucleotide is provided. The method includes the steps of splitting an initiator compound including a functional group operatively linked to a oligonucleotide into multiple fractions; physically immobilizing the initiator compound on a substance; reacting the functional group of the initiator compound with a unique building block to provide the functional moiety; eluting the functional moiety from the substance; reacting the functional moiety with a unique tagging oligonucleotide; optionally, hybridizing the functional moiety with a complementary oligonucleotide; and combining the multiple fractions.

In still another aspect, a method of synthesizing a library of compounds wherein each compound includes a functional moiety including two or more building blocks operatively linked to a tagging oligonucleotide is provided. The method includes (a) splitting a functional moiety operatively linked with an oligonucleotide into multiple fractions; (b) reacting the functional moiety with a unique tagging oligonucleotide; (c) optionally, hybridizing the functional moiety with a complementary oligonucleotide; (d) physically immobilizing the functional moiety on a substance; (e) reacting the functional moiety with a unique building block; (f) eluting the functional moiety from the ion exchange substance; (g) combining the multiple fractions; and optionally, repeating steps a-g, j times wherein j is an integer greater than or equal to 1. In the above method, the functional moiety can be reacted with the unique oligonucleotide tag before reaction of the functional moiety with a building block or the functional moiety can be reacted with a building block before reaction of the functional moiety with the unique oligonucleotide tag.

In still another aspect, a method of identifying a compound from the libraries, supra, which binds to a target is provided. The libraries, supra, are contacted with the target under conditions suitable for at least one compound of the library to bind to the target; compounds of the library that do not bind to the target are removed; the tagging oligonucleotide is amplified; the tagging oligonucleotide is sequenced; and the structure of at least one compound of the library which binds to the target is determined

### DETAILED DESCRIPTION

### Definitions

Unless defined otherwise, all technical and scientific terms used herein have the same meaning as is commonly understood by one of ordinary skill in the art to which this invention belongs. In the event that a plurality of definitions for a term exists, those in this section prevail unless stated otherwise.

It must be noted that as used herein and in the appended claims, the singular forms “a”, “an” and “the” include plural referents unless the context clearly dictates otherwise. Thus, for example, reference to “a tag” includes a plurality of such tags and reference to “the compound” includes reference to one or more compounds and equivalents thereof known to those skilled in the art, and so forth.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

“Building block”, as used herein, is a chemical structural unit which is linked to other chemical structural units or can be linked to other such units. When the functional moiety is polymeric or oligomeric, the building blocks are the monomeric units of the polymer or oligomers. It is to be understood that the term “building block” is used herein to refer to a chemical structural unit as it exists in a functional moiety and also in the reactive form used for the synthesis of the functional moiety. Within the functional moiety, a building block will exist without any portion of the building block which is lost as a consequence of incorporating the budding block into the functional moiety. For example, in cases in which the bond-forming reaction releases a small molecule (see below), the building block as it exists in the functional moiety is a “building block residue”, that is, the remainder of the building block used in the synthesis following loss of the atoms that it contributes to the released molecule.

“Depsipeptide” as used herein refers to a peptide as defined herein where one or more of amide bonds are replaced by ester bonds.

“Functional group” as used herein, refers to a chemical group such as, for example, an electrophilic group, a nucleophilic group, a diene, a dienophile, etc.

“Functional moiety” as used herein, refers to a chemical moiety including one or more building blocks. In some embodiments, the building blocks in the functional moiety are not nucleic acids. The functional moiety can be a linear or branched or cyclic polymer or oligomer or a small organic molecule.

“Linking group or linker” as used herein, is any molecule or substance which performs the function of operatively linking the functional moiety to the tagging oligonucleotide. A linker may vary in structure and length. The linker may be hydrophobic or hydrophilic, long or short, rigid, semirigid or flexible, etc. The linking group can comprise, for example, a polymethylene chain, such as a —(CH₂)ₙ— chain or a polyethylene glycol) chain, such as a —(CH₂CH₂O)ₙ chain, where in both cases n is an integer from 1 to about 20, 5′-O-Dimethoxytrityl-1′,2′-Dideoxyribose-3′-[(2-cyanoethyl)-(N,N-diisopropyl)]-phosphoramidite; 9-O-Dimethoxytrityl-triethylene glycol, 1-[(2-cyanoethyl)-(N,N-diisopropyl)]-phosphoramidite; 3-(4,4′-Dimethoxytrityloxy)propyl-1-[(2-cyanoethyl)-(N,N-diisopropyl)]-phosphoramidite; and 18-O-Dimethoxytritylhexaethyleneglycol,1,-[(2-cyanoethyl)-(N,N-dilsopropyl)]-phosphoramidite, amino-carboxylic linkers (e.g., peptides (e.g., Z-Gly-Gly-Gly-Osu or Z-Gly-Gly-Gly-Gly-Gly-Gly-Osu), PEG (e.g., Fmoc-aminoPEG2000-NHS or amino-PEG (12-24)-NHS), or alkane acid chains (e.g., Boc-ε-aminocaproic acid-Osu)), click chemistry linkers (e.g., peptides (e.g., azidohomalanine-Gly-Gly-Gly-OSu or propargylglycine-Gly-Gly-Gly-OSu), PEG (e.g., azido-PEG-NHS), or alkane acid chains (e.g., 5-azidopentanoic acid, (S)-2-(azidomethyl)-1-Boc-pyrrolidine, or 4-azido-butan-1-oic acid N-hydroxysuccinimide ester)), thiol-reactive linkers (e.g., PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]propionamido)hexanoate))), amidites for oligonucleotide synthesis (e.g., amino modifiers (e.g., 6-(trifluoroacetylamino)-hexyl-(2-cyanoethyl)-(N,N-diisopropyl)-phosphoramidite), thiol modifiers (e.g., S-trityl-6-mercaptohexyl-1-[(2-cyanoethyl)-(N,N-diisopropyl)]-phosphoramidite, or chick chemistry modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamdo)propyl-1-O-succinoyl, long chain alkylamino CPG, or 4-azido-butan-1-oic acid N-hydroxysuccinimide ester)).

“Monolith” as used herein refers to a continuous stationary phase (i.e., a single continuous material (e.g., a polymer or silica base matrix) that contains large interconnected pores or channels allowing high flow rates of mobile phases at moderate pressure.

“Nucleic acid” as used herein refers to an oligonucleotide analog as defined below as well as a double stranded DNA and RNA molecule. A DNA and RNA molecule may include the various analogs defined below.

“Oligonucleotides” or “oligos” as used herein refer to nucleic acid oligomers containing between about 3 and up to about 50, and typically from about 5 to about 30 nucleic acid subunits. In the context of oligos (e.g., hybridization sequence) which direct the synthesis of library compounds, the oligos may include or be composed of naturally-occurring nucleotide residues, nucleotide analog residues, or other subunits capable of forming sequence-specific base pairing, when assembled in a linear polymer, with the proviso that the polymer is capable of providing a suitable substrate for strand-directed polymerization in the presence of a polymerase and one or more nucleotide triphosphates, e.g., conventional deoxyribonucleotides. A “known-sequence oligo” is an oligo whose nucleic acid sequence is known. Oligonucleotides include nucleic acids that have been modified and which are capable of some or all of the chemical or, biological activities of the oligonucleotide from which it was derived. An oligonucleotide analog will generally contain phosphodiester bonds, although in some cases, oligonucleotide analogs are included that may have alternate backbones. Modifications of the ribose-phosphate backbone may facilitate the addition of additional moieties such as labels, or may be done to increase the stability and half-life of such molecules. In addition, mixtures of naturally occurring, nucleic acids and analogs can be made. Alternatively, mixtures of different nucleic acid analogs, and mixtures of naturally occurring nucleic acids and analogs may be made. The oligonucleotides may be single stranded or double stranded, as specified, or contain portions of both double stranded or single stranded sequence. The oligonucleotide may be DNA, RNA or a hybrid, where the nucleic acid contains any combination of deoxyribo-and ribo-nucleotides, and any combination of bases, including uracil, adenine, thymine, cytosine, guanine, inosine, xathanine hypoxathanine, isocytosine, isoguanine, etc.

“Operatively linked”, as used herein, means at least two chemical structures joined together in such a way as to remain linked through the various manipulations described herein. Typically the functional moiety and the encoding oligonucleotide are linked covalently via an appropriate linking group. The linking group is at least a bivalent moiety with a site of attachment for the oligonucleotide and a site of attachment for the functional moiety. For example, when the functional moiety is a polyamide compound, the polyamide compound can be attached to the linking group at its N-terminus, its C-terminus or via a functional group on one of the side chains. The linking group is sufficient to separate the polyamide compound and the oligonucleotide by at least one atom and in some embodiments by more than one atom. In some embodiments, the linking group is sufficiently flexible to allow the polyamide compound to bind target molecules in a manner which is independent of the oligonucleotide.

“Peptide” as used herein refers to a polymer of amino acid residues between about 2 and 50 amino acid residues, between about 2 and 20 amino acid residues, or between about 2 and 10 residues. Peptides include modified peptides such as, for example, glycopeptides, PEGylated peptides, lipopeptides, peptides conjugated with organic or inorganic ligands, peptides which contain peptide bond isosteres (e.g., ψ[CH₂S], ψ[CH₂NH₂], ψ[NHCO], ψ[COCH₂], ψ[(E) or (Z) CH═CH], etc and also include cyclic peptides. In some embodiments, the amino acid residues may be any L-α-amino acid, D-α-amino residue, N-alkyl variants thereof or combinations thereof. In other embodiments, the amino acid residues may any L-α-amino acid, D-α-amino residue, β-amino acids, γ-amino acids, N-alkyl variants thereof or combinations thereof.

“Peptoid” as used herein refers to polymers of poly N-substituted glycine (Simon et al., Proc. Natl. Acad. Sci. (1992) 89(20) 9367-9371) and include cyclic variants thereof.

“Polypeptide” as used herein refers to a polymer of amino acid residues typically comprising greater than 50 amino acid residues and includes cyclic variants thereof. Polypeptide includes proteins (including modified proteins such as glycoproteins, PEGylated proteins, lipoproteins, polypeptide conjugates with organic or inorganic ligands, etc.) receptor, receptor fragments, enzymes, structural proteins (e.g., collagen) etc. In some embodiments, the amino acid residues may be any L-α-amino acid, D-α-amino residue, or combinations thereof. In other embodiments, the amino acid residues may be any L-α-amino acid, D-α-amino residue, N-alkyl variants thereof or combinations thereof.

“Polymer” includes copolymers, and the term “monomer” includes co-monomers. Polymers include, for example, polyamides, phospholipids, polycarbonates, polysaccharides, polyurethanes, polyesters, polyureas, polyacetates, polyarylene sulfides, polyethylenimines, polyimides, etc.

Reference will now be made in detail to embodiments of the invention. While the invention will be described in conjunction with these embodiments, it will be understood that it is not intended to limit the invention to the embodiments, infra. To the contrary, it is intended to cover alternatives, modifications, and equivalents as may be included within the spirit and scope of the invention as defined by the appended claims.

### Methods of Synthesis of Oligonucleotide Tagged Combinatorial Libraries and Uses Thereof

The present invention satisfies these and other needs by providing methods for synthesis of a compound which includes a functional moiety operatively linked to a tagging oligonucleotide, libraries thereof and methods of using the compound and libraries thereof to identify compounds which bind to a target.

A method of synthesizing a compound including a functional moiety operatively linked to a tagging oligonucleotide is provided. The method includes the steps of splitting an initiator compound including a functional group operatively linked to a tagging oligonucleotide into multiple fractions; reacting the initiator compound with a unique oligonucleotide tag; optionally, hybridizing the initiator compound with a complementary oligonucleotide; non-covalently immobilizing the initiator compound on a substance; reacting the functional group of the initiator compound with a unique building block to provide the functional moiety; eluting the functional moiety from the substance; and combining the multiple fractions.

Another method of synthesizing a compound including a functional moiety operatively linked to a tagging oligonucleotide is provided. The method includes the steps of splitting an initiator compound including a functional group operatively linked to a tagging oligonucleotide into multiple fractions; optionally, hybridizing the initiator compound with a complementary oligonucleotide; non-covalently immobilizing the initiator compound on a substance; reacting the functional group of the initiator compound with a unique building block to provide the functional moiety; eluting the functional moiety from the substance; and combining the multiple fractions.

Still another method of synthesizing a compound including a functional moiety operatively linked to a tagging oligonucleotide is provided. The method includes the steps of splitting an initiator compound including a functional group operatively linked to a oligonucleotide into multiple fractions; non-covalently immobilizing the initiator compound on a substance; reacting the functional group of the initiator compound with a unique building block to provide the functional moiety; eluting the functional moiety from the substance; reacting the functional moiety with a unique tagging oligonucleotide; optionally, hybridizing the functional moiety with a complementary oligonucleotide; and combining the multiple fractions.

Still another method of synthesizing a library of compounds wherein each compound includes a functional moiety including two or more building blocks operatively linked to a tagging oligonucleotide is provided. The method includes (a) splitting a functional moiety operatively linked with an oligonucleotide into multiple fractions; (b) reacting the functional moiety with a unique tagging oligonucleotide; (c) optionally, hybridizing the functional moiety with a complementary oligonucleotide; (d) non-covalently immobilizing the functional moiety on a substance; (e) reacting the functional moiety with a unique building block; (f) eluting the functional moiety from the ion exchange substance; (g) combining the multiple fractions; and optionally, repeating steps a-g, j times wherein j is an integer greater than or equal to 1. In the above method, the functional moiety can be reacted with the unique oligonucleotide tag before reaction of the functional moiety with a building block or the functional moiety can be reacted with a building block before reaction of the functional moiety with the unique oligonucleotide tag.

Still another method of synthesizing a compound including a functional moiety operatively linked to a tagging oligonucleotide is provided. The method includes the steps of splitting an initiator compound including a functional group operatively linked to a tagging oligonucleotide into multiple fractions; reacting the initiator compound with a unique oligonucleotide tag; optionally, hybridizing the initiator compound with a complementary oligonucleotide; covalently immobilizing the initiator compound on a substance; reacting the functional group of the initiator compound with a unique building block to provide the functional moiety; eluting the functional moiety from the substance; and combining the multiple fractions.

Still another method of synthesizing a compound including a functional moiety operatively linked to a tagging oligonucleotide is provided. The method includes the steps of splitting an initiator compound including a functional group operatively linked to a tagging oligonucleotide into multiple fractions; optionally, hybridizing the initiator compound with a complementary oligonucleotide; covalently immobilizing the initiator compound on a substance; reacting the functional group of the initiator compound with a unique building block to provide the functional moiety; eluting the functional moiety from the substance; and combining the multiple fractions.

Still another method of synthesizing a compound including a functional moiety operatively linked to a tagging oligonucleotide is provided. The method includes the steps of splitting an initiator compound including a functional group operatively linked to a oligonucleotide into multiple fractions; covalently immobilizing the initiator compound on a substance; reacting the functional group of the initiator compound with a unique building block to provide the functional moiety; eluting the functional moiety from the substance; reacting the functional moiety with a unique tagging oligonucleotide; optionally, hybridizing the functional moiety with a complementary oligonucleotide; and combining the multiple fractions.

Another method of synthesizing a library of compounds wherein each compound includes a functional moiety including two or more building blocks operatively linked to a tagging oligonucleotide is provided. The method includes (a) splitting a functional moiety operatively linked with an oligonucleotide into multiple fractions; (b) reacting the functional moiety with a unique tagging oligonucleotide; (c) optionally, hybridizing the functional moiety with a complementary oligonucleotide; (d) covalently immobilizing the functional moiety on a substance; (e) reacting the functional moiety with a unique building block; (f) eluting the functional moiety from the ion exchange substance; (g) combining the multiple fractions; and optionally, repeating steps a-g, j times wherein j is an integer greater than or equal to 1. In the above method, the functional moiety can be reacted with the unique oligonucleotide tag before reaction of the functional moiety with a building block or the functional moiety can be reacted with a building block before reaction of the functional moiety with the unique oligonucleotide tag.

Still another method of synthesizing a compound including a functional moiety operatively linked to a tagging oligonucleotide is provided. The method includes the steps of splitting an initiator compound including a functional group operatively linked to a tagging oligonucleotide into multiple fractions; reacting the initiator compound with a unique oligonucleotide tag; optionally, hybridizing the initiator compound with a complementary oligonucleotide; physically immobilizing the initiator compound on a substance; reacting the functional group of the initiator compound with a unique building block to provide the functional moiety; eluting the functional moiety from the substance; and combining the multiple fractions.

Still another method of synthesizing a compound including a functional moiety operatively linked to a tagging oligonucleotide is provided. The method includes the steps of splitting an initiator compound including a functional group operatively linked to a tagging oligonucleotide into multiple fractions; optionally, hybridizing the initiator compound with a complementary oligonucleotide; physically immobilizing the initiator compound on a substance; reacting the functional group of the initiator compound with a unique building block to provide the functional moiety; eluting the functional moiety from the substance; and combining the multiple fractions.

Still another method of synthesizing a compound including a functional moiety operatively linked to a tagging oligonucleotide is provided. The method includes the steps of splitting an initiator compound including a functional group operatively linked to a oligonucleotide into multiple fractions; physically immobilizing the initiator compound on a substance; reacting the functional group of the initiator compound with a unique building block to provide the functional moiety; eluting the functional moiety from the substance; reacting the functional moiety with a unique tagging oligonucleotide; optionally, hybridizing the functional moiety with a complementary oligonucleotide; and combining the multiple fractions.

Still another method of synthesizing a library of compounds wherein each compound includes a functional moiety including two or more building blocks operatively linked to a tagging oligonucleotide is provided. The method includes (a) splitting a functional moiety operatively linked with an oligonucleotide into multiple fractions; (b) reacting the functional moiety with a unique tagging oligonucleotide; (c) optionally, hybridizing the functional moiety with a complementary oligonucleotide; (d) physically immobilizing the functional moiety on a substance; (e) reacting the functional moiety with a unique building block; (f) eluting the functional moiety from the ion exchange substance; (g) combining the multiple fractions; and optionally, repeating steps a-g, j times wherein j is an integer greater than or equal to 1. In the above method, the functional moiety can be reacted with the unique oligonucleotide tag before reaction of the functional moiety with a building block or the functional moiety can be reacted with a building block before reaction of the functional moiety with the unique oligonucleotide tag.

A method of identifying a compound from the libraries, supra, which binds to a target, is provided. The libraries, supra, is contacted with the target under conditions suitable for at least one compound of the library to bind to the target; compounds of the library that do not bind to the target are removed; the tagging oligonucleotide is amplified; the tagging oligonucleotide is sequenced; and the structure of at least one compound of the library which binds to the target is determined.

In some embodiments of the methods described above, where the functional moiety or initiator compound is non-covalently immobilized, the substance is an ion exchange substance. In other embodiments, the ion exchange substance is a monolith, a resin, a filter, a polyelectrolyte compound or hydroxyapatite. In some of these embodiments, the functional moiety is eluted with a salt solution.

In some embodiments of the methods described above where the functional moiety or initiator compound is non-covalently immobilized, the substance is reversed phase substance. In some embodiments, the reversed phase substance is C₈ or C₁₈ silica or C₈ or C₁₈ sepharose resin. In some of these embodiments, the functional moiety is eluted with a polar organic solvent.

In some embodiments of the methods described above, where the functional moiety or initiator compound is covalently immobilized, the substance is a monolith, a resin, a magnetic bead, a bead, a magnetic bead, a compound or a dendrimer. The functional moiety or initiator compound may be covalently attached and/or cleaved from the substance by chemical or enzymatic means.

In some embodiments of the methods described above where the functional moiety or initiator compound is covalently immobilized, disulfide, ester, amide or nucleotide bonds attach the functional moiety or initiator compound to the substance. In other embodiments, reduction of a disulfide bond is used to cleave the functional moiety or initiator compound from the substance. In still other embodiments, hydrolysis of an ester, amide or nucleotide bond is used to cleave the functional moiety or initiator compound from the substance.

In some embodiments of the methods described above, where the functional moiety or initiator compound is physically immobilized, the substance is a nanoparticle, filter paper or an electroconducting, an ultrafiltration, or size exclusion material. In some of these embodiments, the material is a gel or a membrane. In some of these embodiments, the functional moiety or initiator compound is removed from the substance by ultracentrifugation, elution or dissolution in solvent followed by isolation and concentration of the solution.

In any of the methods of synthesis of compounds and/or libraries of compounds, described above, the functional moiety is operatively linked to the tagging oligonucleotide with a linking group. Generally, the linking group is at least bivalent with a site of attachment for the tagging oligonucleotide and a site of attachment for the functional moiety. In some embodiments, the linking group separates the tagging oligonucleotide and the functional moiety by between about one atom and about one hundred atoms. In other embodiments, the linking group separates the tagging oligonucleotide and the functional moiety by between about five atoms and about fifty atoms. In still other embodiments, the linking group separates the tagging oligonucleotide and the functional moiety by between about ten atoms and about thirty atoms. In some embodiments, the linking group separates the tagging oligonucleotide and the functional moiety by greater than about 10 Å. In other embodiments, the linking group separates the tagging oligonucleotide and the functional moiety by greater than about 50 Å. In still other embodiments, the linking group separates the tagging oligonucleotide and the functional moiety by greater than about 100 Å.

In some embodiments, the linking group is attached to the initiator compound or functional moiety and the 5′-phosphate group of the oligonucleotide. In other embodiments, the linking group is attached to the initiator compound or functional moiety and the 3′-phosphate group of the oligonucleotide. For example, the linking group can be derived from a linking group precursor comprising an activated carboxyl group on one end and an activated ester on the other end. Reaction of the linking group precursor with the N-terminal nitrogen atom will form an amide bond connecting the linking group to the polyamide compound or N-terminal building block, while reaction of the linking group precursor with the 5′-hydroxy group of the oligonucleotide will result in attachment of the oligonucleotide to the linking group via an ester linkage. The linking group can comprise many types of compounds such as, for example, a polymethylene chain, such as a —(CH₂)ₙ— chain or a poly(ethylene glycol) chain, such as a —(CH₂CH₂O)ₙ chain, where in both cases n is an integer from 1 to about 20.

In any of the methods of synthesis of compounds and/or libraries of compounds described above, the functional group is complementary to a functional group in the building block as defined infra.

Reacting the functional moiety or an initiator compound with a unique tagging oligonucleotide includes both chemical and enzymatic methods including both enzymatic and chemical ligation (Litovchick et al., Artif DNA PBA 2014; 5: e27896, Morgan et al., U.S. Pat. No. 7,972,992; Morgan et al., U.S. Pat. No. 7,935,658; Morgan et al., U.S. Patent Application No. 2011/0136697; Morgan et al., U.S. Pat. No. 7,972,994; Morgan et al., U.S. Pat. No. 7,989,395; Morgan et al., U.S. Pat. No. 8,410,028; Morgan et al., U.S. Pat. No. 8,598,089; Morgan et al., U.S. patent application Ser. No. 14/085,271; Wagner et al., U.S. Patent Application No. 2012/0053901; and Keefe et al., U.S. Patent Application No. 2014/0315762).

The building blocks used in any of the methods of synthesis of compounds and/or libraries of compounds described above, can be any chemical compounds which are complementary, that is the building blocks must be able to react together to form a structure comprising two or more building blocks. Typically, all of the building blocks used will have at least two reactive functional groups, although it is possible that some of the building blocks (for example the last building block in an oligomeric functional moiety) used will have only one reactive group each. Reactive groups on two different building blocks should be complementary, i.e., capable of reacting together to form a covalent bond, optionally with the concomitant loss of a small molecule, such as water, HCl, HF and so forth.

Two reactive functional groups are complementary if they are capable of reacting together to form a covalent bond. In some embodiments, the bond forming reactions occur rapidly under ambient conditions without substantial formation of side products. In other embodiments, given reactive functional group will react with a given complementary reactive functional group exactly once. In still other embodiments, complementary reactive groups of two building blocks react, for example, via nucleophilic substitution, to form a covalent bond.

In some embodiments, one member of a pair of complementary reactive functional groups is an electrophilic group and the other member of the pair is a nucleophilic group. Complementary electrophilic and nucleophilic groups include any two groups which react via nucleophilic substitution under suitable conditions to form a covalent bond as is well known to the skilled artisan. A variety of suitable bond-forming reactions are known in the art. Examples of suitable electrophilic groups include reactive carbonyl groups, such as acyl chloride groups, ester groups, including carbonyl pentafluorophenyl esters and succinimide esters, ketone groups and aldehyde groups; reactive sulfonyl groups, such as sulfonyl chloride groups, and reactive phosphonyl groups. Other electrophilic groups include, for example, terminal epoxide groups, isocyanate groups and alkyl halide groups. Suitable nucleophilic groups include, for example, primary and secondary amino groups and hydroxyl groups and carboxyl groups. Suitable complementary reactive groups are set forth below. One of skill in the art can readily determine other reactive group pairs that can be used in the present method, and the examples provided herein are not intended to be limiting.

In some embodiments, the complementary reactive functional groups include activated carboxyl groups, reactive sulfonyl groups or reactive phosphonyl groups, or a combination thereof, and primary or secondary amino groups. In these embodiments, the complementary reactive groups react under suitable conditions to form an amide, sulfonamide or phosphonamidate bond.

In other embodiments, the complementary reactive functional groups include epoxide groups and primary or secondary amino groups. An epoxide-containing building block reacts with an amine-containing building block under suitable conditions to form a carbon-nitrogen bond, resulting in a amino alcohol. In still other embodiments, the complementary reactive groups include aziridine groups and primary or secondary amino groups. Under suitable conditions, an aziridine-containing building block reacts with an amine-containing building block to form a carbon-nitrogen bond, resulting in a 1,2-diamine.

In still other embodiments, the complementary reactive functional groups include isocyanate groups and primary or secondary amino groups. An isocyanate-containing building block will react with an amino-containing building block under suitable conditions to form a carbon-nitrogen bond, resulting in a urea group.

In still other embodiments, the complementary reactive functional groups include isocyanate groups and hydroxyl groups. An isocyanate-containing building block will react with a hydroxyl-containing building block under suitable conditions to form a carbon-oxygen bond, resulting in a carbamate group.

In still other embodiments, the complementary reactive functional groups include amino groups and carbonyl-containing groups, such as aldehyde or ketone groups. Amines react with such groups via reductive amination to form a new carbon-nitrogen bond.

In still other embodiments, the complementary reactive functional groups include phosphorous ylide groups and aldehyde or ketone groups, A phosphorus-ylide-containing building block will react with an aldehyde or ketone-containing building block under suitable conditions to form a carbon-carbon double bond, resulting in an alkene.

In still other embodiments, the complementary reactive functional groups react cycloaddition to form a cyclic structure. One example of such complementary reactive groups are alkynes and organic azides, which react under suitable conditions to form a triazole ring structure.

In still other embodiments, the complementary reactive functional groups are an alkyl halide and a nucleophile, such as an amino group, a hydroxyl group or a carboxyl group. Such groups react under suitable conditions to form a carbon-nitrogen (alkyl halide plus amine) or carbon oxygen (alkyl halide plus hydroxyl or carboxyl group).

In still other embodiments, the complementary reactive functional groups are a halogenated heteroaromatic group and a nucleophile, and the building blocks are linked under suitable conditions via aromatic nucleophilic substitution. Suitable halogenated heteroaromatic groups include chlorinated pyrimidines, triazines and purines, which react with nucleophiles, such as amines, under mild conditions in aqueous solution.

It is to be understood that the synthesis of a functional moiety described in the methods disclosed above can proceed via one particular type of coupling reaction, such as, but not limited to, one of the reactions discussed above, or via a combination of two or more coupling reactions, such as two or more of the coupling reactions discussed above. For example, in some embodiments, the building blocks are joined by a combination of amide bond formation (amino and carboxylic acid complementary groups) and reductive amination (amino and aldehyde or ketone complementary groups). Any coupling chemistry can be used, provided that it is compatible with the presence of an oligonucleotide.

A building block used in any of the methods of synthesis described above can include one or more functional groups in addition to the reactive group or groups employed to form the functional moiety. One or more of these additional functional groups can be protected to prevent undesired reactions of these functional groups. Suitable protecting groups are known in the art for a variety of functional groups.

In some embodiments, the building block used in any of the methods of synthesis described above is a monomer. In other embodiments, the building block is a amino acid, a N-alkyl amino acid, a L-α-amino acid, a D-α-amino residue, a β-amino acid, a γ-amino acid, a hydroxy acid, a peptide, a nucleic acid, a polypeptide, a depsipeptide, a oligonucleotide, a hydroxy phosphonic acid, a amino sulfonic acid, a hydroxysulfonic acid, a N-substituted glycine, a organic compound with two or more functional groups, a inorganic compound with two or more functional groups or combinations thereof. In still other embodiments, the building block is modified after incorporation into the functional moiety.

In some embodiments, the functional moiety provided by any of the methods of synthesis described above is a polymer. In other embodiments, the functional moiety is a peptide, a depsipeptide, a nucleic acid, a polypeptide, a inorganic compounds of molecular weight greater that 50 daltons, a organic compounds of molecular weight between about 3000 daltons and about 50 daltons or combinations thereof.

The tagging oligonucleotide used in any of the methods of synthesis described above can be of any desirable length, but is usually at least three nucleobases in length. In some embodiments, the tagging oligonucleotide is 4 or more nucleobases in length. In other embodiments, the tagging oligonucleotide is from 3 to about 30 nucleobases in length. In still other embodiments, the tagging oligonucleotide is from 3 to about 12 nucleobases in length. In still other embodiments, the tagging oligonucleotides of the molecules in the libraries described above have a common terminal sequence which can serve as a primer for PCR, as is known in the art. Such a common terminal sequence can be incorporated as the terminal end of the incoming oligonucleotide added in the final cycle of the library synthesis, or it can be added following library synthesis, for example, using enzymatic or chemical ligation methods or chemical synthesis.

In the method of identifying a compound from the library which binds to a target disclosed above, the tagging nucleotide is amplified by polymerase chain reaction, linear chain reaction or rolling circle amplification. Such methods are well known in the art and within the ambit of the skilled artisan. The amplified nucleic acid may be synthesized by well known sequencing methods, including, but not limited to, Next Gen Sequencing, which are also known to those of skill in the art.

Generally, the target can be any substance, including any molecule, for which identification of compounds with affinity is desirable. In some embodiments, the target is a biological molecule. In other embodiments, the target is an enzyme, a receptor, an ion channel, a nucleic acid, a carbohydrate, protein-protein interface, a virus, bacteria, a eukaryotic cell or a prion.

Compounds and/or libraries synthesized by the methods disclosed above, can in certain embodiments, serve as starting materials, in the method disclosed by Harbury (Harbury et al., U.S. Pat. No. 7,479,472). Furthermore, many of the methods known in the art for preparing oligonucleotide tagged compounds and/or libraries can also be used as starting material for the construction of DNA-templated combinatorial libraries in the method disclosed by Harbury (Harbury et al., U.S. Pat. No. 7,479,472).

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims.

All publications and patents cited herein are incorporated by reference in their entirety to disclose and describe the methods and/or materials in connection with which the publications are cited. It is understood that the present disclosure supersedes any disclosure of an incorporated publication to the extent there is a contradiction. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. A method of synthesizing a library of compounds wherein each compound comprises a functional moiety comprising two or more building blocks operatively linked to a tagging oligonucleotide:
a. splitting a functional moiety operatively linked with an oligonucleotide into multiple fractions;
b. reacting the functional moiety with a unique tagging oligonucleotide;
c. optionally, hybridizing the functional moiety with a complementary oligonucleotide;
d. non-covalently immobilizing the compound of step b or step c on a substance;
e. reacting the functional moiety with a unique building block;
f. eluting the functional moiety from the substance;
g. combining the multiple fractions; and
h. optionally, repeating steps a-g, j times wherein j is an integer greater than or equal to 1.

2. A method of identifying a compound which binds to a target comprising:
a. acting the library of claim 1 with the target under conditions suitable for at least one compound of the library to bind to the target;
b. removing compounds of the library that do not bind to the target;
c. amplifying the tagging oligonucleotide;
d. sequencing the tagging oligonucleotide of step (c); and
e. determining the structure of at least one compound of the library which binds to the target.

3. A method of synthesizing a library of compounds wherein each compound comprises a functional moiety comprising two or more building blocks operatively linked to a tagging oligonucleotide:
a. splitting a functional moiety operatively linked with an oligonucleotide into multiple fractions;
b. reacting the functional moiety with a unique tagging oligonucleotide;
c. optionally, hybridizing the functional moiety with a complementary oligonucleotide;
d. covalently immobilizing the compound of step b or step c on a substance;
e. reacting the functional moiety with a unique building block;
f. eluting the functional moiety from the substance;
g. combining the multiple fractions; and
h. optionally, repeating steps a-g, j times wherein j is an integer greater than or equal to 1.

4. A method of identifying a compound which binds to a target comprising:
a. acting the library of claim 3 with the target under conditions suitable for at least one compound of the library to bind to the target;
b. removing compounds of the library that do not bind to the target;
c. amplifying the tagging oligonucleotide;
d. sequencing the tagging oligonucleotide of step (c); and
e. determining the structure of at least one compound of the library which binds to the target.

5. A method of synthesizing a library of compounds wherein each compound comprises a functional moiety comprising two or more building blocks operatively linked to a tagging oligonucleotide:
a. splitting a functional moiety operatively linked with an oligonucleotide into multiple fractions;
b. reacting the functional moiety with a unique tagging oligonucleotide;
c. optionally, hybridizing the functional moiety with a complementary oligonucleotide;
d. physically immobilizing the compound of step b or step c on a substance;
e. reacting the functional moiety with a unique building block;
f. eluting the functional moiety from the substance;
g. combining the multiple fractions; and
h. optionally, repeating steps a-g, j times wherein j is an integer greater than or equal to 1.

6. A method of identifying a compound which binds to a target comprising:
a. acting the library of claim 5 with the target under conditions suitable for at least one compound of the library to bind to the target;
b. removing compounds of the library that do not bind to the target;
c. amplifying the tagging oligonucleotide;
d. sequencing the tagging oligonucleotide of step (c); and
e. determining the structure of at least one compound of the library which binds to the target.
